(19) United States
(12) Patent Application Publication
(10) Pub. No.: US 2010/0312352 A1
(43) Pub. Date: Dec. 9, 2010

(54) METHOD FOR COATING PROSTHESIS ELEMENTS AND ELEMENTS OBTAINABLE WITH THE METHOD

(76) Inventors: Nelso Antolotti, Collecchio Di Parma (IT); Emanuele Magalini, Trento (IT)

Correspondence Address: TUTUNJIAN & BITETTO, P.C., 20 CROSSWAYS PARK NORTH, SUITE 210, WOODBURY, NY, 11797, US

(21) Appl. No.: 12/677,970

(22) PCT Filed: Sep. 1, 2008
(86) PCT No.: PCT/IB08/02261

§ 371 Date: Mar. 12, 2010

(30) Foreign Application Priority Data
Sep. 12, 2007 (IT) MO2007A000260

(51) Int. Cl. A61F 2/32; B05D 7/00; B05D 5/00

(52) U.S. Cl. 623/22.11; 427/2.24; 427/2.26

(57) ABSTRACT

A method for coating prosthesis elements that have a concave surface and a corresponding convex counter surface includes arranging in at least the concave surface a shaped retention surface for gripping a coating. The coating is coupled with the shaped retention surface such that coupling includes coating the concave surface with the coating temporarily in a moldable state to obtain a moldable coating. The moldable coating is made to solidify and adhere firmly to the shaped retention surface.

### TECHNICAL FIELD

The invention relates to a method for coating prosthesis elements and elements obtainable with the method, particularly suitable for the coupling of resistant-to-wear polymer components and metal components for articulate prostheses, in detail for making prostheses for replacing the joint of a hip.

### STATE OF THE ART

Prostheses are known for replacing the hip joint in patients suffering from serious illnesses, such as, for example, arthrosis or arthritis, or who have suffered fractures that do not permit spontaneous bone reconstruction by simple immobilisation of the pelvis and of the thigh bone.

These prostheses comprise a first portion that must be implanted in a longitudinal direction in the thigh bone and which forms an end turned towards the pelvis shaped like a sphere or the like, which practically reproduces the head of the thigh bone, and a second portion shaped like a cup and called cotyle, which is arranged in the cotyloid cavity of the hip, meaning at the point where the head of the thigh bone engages in this, forming the joint that makes possible the movements of the leg.

Because this joint has to withstand all the loads transmitted to the pelvis by the legs, the prostheses must have a limited friction between the two components in reciprocal contact and must be very strong and fixable to the respective bone portions in a stable way, without any micro movements occurring between one of the components and the bone portion to which this is associated: in fact, these micro movements are very damaging for the life span of the prosthesis.

Over time, in fact, these micro movements can determine the occurrence of clearance between one of the components of the prosthesis and the bone part to which it is fitted; this clearance tends to gradually increase over time, until these components, normally the cotyle, become detached from the bone portion, in this case the cotyloid cavity of the hip, to which it has been fixed by means of a surgical operation.

In particular, the cotyle, meaning the portion of the prosthesis that has to be fitted inside the cotyloid cavity, and ready to receive the spherical head of the other portion of the prosthesis fixed to the thigh bone, must not only be mechanically very strong to withstand the loads determined by walking, but also very lightweight so as not to create problems for the patient after implantation and, above all, biologically compatible, to prevent any rejection on the part of the body.

Normally, known cotyles can be made of titanium, steel, stellite (a cobalt-chrome alloy) and their alloys, or of high-resistance ceramic and polymer plastic materials.

Furthermore, the convex surface is treated in such a way as to show roughness distributed on this that favour the gripping interaction between this and the inside of the cotyloid cavity and maintenance of the immobility of the cotyle in this cavity, after implantation.

This roughness can be obtained by means of a mechanical operation on the outer surface of the cotyle, for example, by means of sandblasting treatments, or attack of this surface by chemical substances or, again, by depositing on this surface rough coverings applied from outside using known techniques such as, for example, the Plasma Spray technique.

In the concave portion on the other hand is arranged, normally by means of a press-fitting, an anti-friction material coating layer with a predetermined thickness and which allows the spherical head of the other side of the prosthesis to be fitted and to rotate inside this without appreciable friction.

This coating layer is first of all created according to a specific design in a mould specially prepared for this purpose, or else by means of mechanical machining on pre-forms and then, after forming, it is fitted and blocked inside the concave surface of the cotyle.

This state of the art does however have a number of drawbacks.

A first drawback is that because of the considerable thickness of the coating of the cotyle concavity, the diameter of the spherical head of the other side of the prosthesis must necessarily be limited.

Another drawback is that this limitation of the diameter of the spherical head on the one hand limits the overall strength of the prosthesis due to a reduced supporting surface between the spherical head of the thigh bone and the cotyle concavity and, finally, the amount of load the prosthesis can withstand and, on the other hand, considerably increases the friction between the two surfaces in reciprocal contact, determining substantially quick wear of the cotyle coating and the risk of the prosthesis becoming blocked.

A further drawback is that the diameter of the cotyle cannot be increased in order to also proportionately increase the diameter of the internal cavities of the cotyle in order to use heads of thigh bones with correspondingly larger diameters, because a cotyle with dimensions larger than those of known cotyles would be very hard to fit inside the cotyloid cavity and, in any case, an excessive diameter of this cotyle could stress the walls of the cotyloid cavity during fitting, even causing, at worst, the walls to fracture.

Another drawback is that the components of some of the known prostheses are too rigid and this results in the stresses made during walking being discharged onto the components of the prosthesis and not also distributed on the bone part to which the latter are fitted.

Consequently, the bone part, not being adequately stressed or being stressed to a small extent, tends to gradually reduce in volume, generating clearances over time, and, therefore, relevant movements between this and the parts making up the prosthesis.

This, with the passing of time, makes the fitting of the latter pointless.

### OBJECTS OF THE INVENTION

One object of the invention is to upgrade the state of the art.

Another object of the invention is to create a method for coating prosthesis elements and elements obtainable with the method that allow making prostheses that are strong and lightweight.

A further object of the invention is to create a method for coating prosthesis elements that allows making prostheses that do not generate movements between these and the bone parts to which they are applied or with which they are in contact.

Another object of the invention is to make prostheses that have contact and jointing surfaces with the bone tissue having high roughness and obtainable directly during forming, for example using so-called “Rapid Manufacturing” techniques.

Another object of the invention is to create a method for coating prosthesis elements and elements obtainable with the method that allow obtaining prostheses able to keep the bone parts to which they are applied under stress, preventing the harmful shrinking of such parts.

According to one aspect of the invention, a method is provided for coating prosthesis elements that have a concave surface and a corresponding convex counter surface, comprising: arranging in at least said concave surface retentive means for gripping coating means; coupling coating means with said retentive means, characterized in that said coupling comprises coating said concave surface with said coating means being in a state chosen between liquid, fluid, pasty; making said coating means and said retentive means adhere together.

According to another aspect of the invention, a prosthesis element is provided obtainable, with a method for coating prosthesis elements characterized in that it has: a concave surface; a layer of low friction coating material arranged on said concave surface and which can be coupled with a natural or artificial joined convex surface of, or shaping, a bone body, said coating layer being penetrated by retentive means obtained on said concave surface.

The method for coating prosthesis elements and the prosthesis elements obtainable with the method for coating prosthesis elements therefore permit:
making prostheses for replacing the hip joint that remain firmly fastened to the bone parts to which they are applied;
making prostheses that are substantially lightweight;
to make prostheses that have an excellent joint between the head of the portion to be applied on the thigh bone and the portion (cotyle) to be fitted in the cotyloid cavity of the hip; and
to make prostheses that allow discharging the stress transmitted by the legs onto the bone tissue as well, keeping this active and therefore voluminous enough so as not to create, over time, reductions in volume and the consequent occurrence of clearances between the prosthesis and the replaced bone portion.

### BRIEF DESCRIPTION OF THE DRAWINGS

Further characteristics and advantages of the invention will appear more evident from the detailed description of a prosthesis element obtainable with a method for coating prosthesis elements, illustrated indicatively by way of non limiting example, in the attached drawings wherein:

FIG. 1 is an interrupted and partially section schematic view of a hip prosthesis;

FIG. 2 is an interrupted and section schematic view of a final phase of the forming of a coating of the internal cavity of a cotyle to make the hip prosthesis of FIG. 1;

FIG. 3 is an interrupted view on an enlarged scale of a detail of FIG. 2;

FIG. 4 is an interrupted and section schematic view of a forming phase of a coating of the internal cavity of a cotyle to make the hip prosthesis of FIG. 1;

FIG. 5 is a perspective view of a cotyle to make the prosthesis of FIG. 1, in a first possible version;

FIG. 6 is a cross section of the cotyle of FIG. 5, according to a VI-VI track plane;

FIG. 7 is an interrupted view of an enlarged detail of the cross section of FIG. 6;

FIG. 8 is a perspective view of a second version of a cotyle to make the prosthesis of FIG. 1;

FIG. 9 is a perspective view of a detail on a much enlarged scale of the second version of the cotyle of FIG. 8;

FIG. 10 is a perspective view of a portion of a third version of a cotyle to make the prosthesis of FIG. 1;

FIG. 11 is an interrupted view of a detail on a much enlarged scale of the cotyle of FIG. 10;

FIG. 12 is a perspective view of a fourth version of a cotyle to make the prosthesis of FIG. 1;

FIG. 13 is an interrupted view of a portion of the cotyle of FIG. 12, on an enlarged scale;

FIG. 14 is an interrupted side view and on a further enlarged scale of the portion of the cotyle of FIG. 13;

FIG. 15 is a perspective view of a fifth version of a cotyle to make the prosthesis of FIG. 1;

FIG. 16 is an interrupted view of a portion of the cotyle of FIG. 15, on an enlarged scale;

FIG. 17 is an interrupted side view and on a further enlarged scale of the portion of the cotyle of FIG. 16;

FIG. 18 is a cross section view of a sixth version of a cotyle to make the prosthesis of FIG. 1;

FIG. 19 is an interrupted view and on an enlarged scale of a portion of the cotyle of FIG. 18;

FIG. 20 is a perspective view of a seventh version of a cotyle to make the prosthesis of FIG. 1;

FIG. 21 is a section view of the cotyle of FIG. 20, taken according to a XXII-XXII plane;

FIG. 22 is an interrupted view and on an enlarged scale of a detail of the section of the cotyle of FIG. 21;

FIG. 23 is a perspective view of a eighth version of a cotyle to make the prosthesis of FIG. 1;

FIG. 24 is a section view of the cotyle of FIG. 23, taken according to a XXIV-XXIV track plane;

FIG. 25 is an interrupted view and on enlarged scale of a detail of the section of the cotyle of FIG. 24.

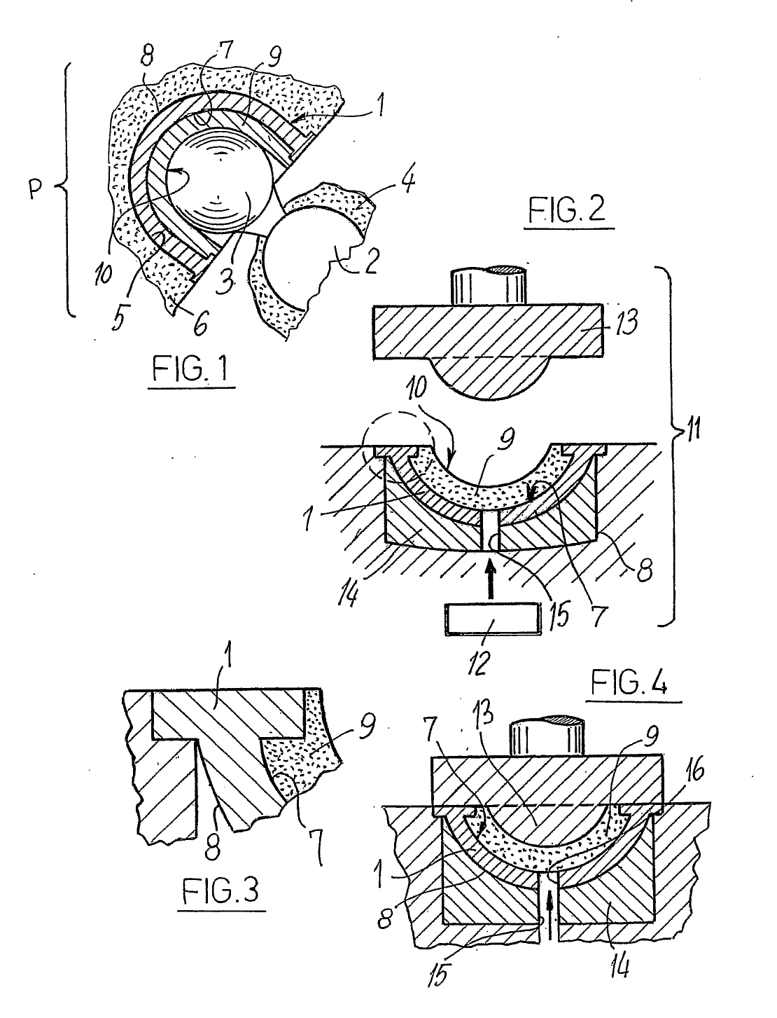

### EMBODIMENTS OF THE INVENTION

With reference to the Figures, by 1 is generically indicated a cup cotyle, normally shaped like a sphere, to form a prosthesis “P”, which also comprises a portion 2 having a spherical head 3, or substantially such, and which can be applied on a thigh bone 4, which can be coupled with the cotyle 1 to replace the joint of a hip.

The cotyle 1, which is to be fitted and fixed inside the cotyloid cavity 5 of a hip 6, defines a concave surface 7 and a convex counter surface 8.

The concave surface 7 can be coated with a layer 9 of polymer material which defines a face 10, also concave, with a low friction coefficient and which is to come into contact with the spherical head 3 of the portion 2.

To make the layer 9, the polymer material is prepared in such a state as to make it mouldable, meaning, in effect, in a state chosen from liquid, fluid or pasty, so that it can shape itself automatically on the concave surface 7.

According to the method for coating prosthesis elements “P”, the polymer material is injected inside a mould 11 by means of a known injection device 12. The mould 11 comprises a convex punch 13 and a concave die 14; the profiles of the convex punch 13 and of the concave die 14 are substantially complementary to each other, but have dimensions such as to define an inter-space between them when they are coupled together.

Inside the inter-space, the cotyle 1 can be arranged, the concave surface 7 of which is to be coated, as shown in the FIGS. 2 and 4 and, between this and the convex punch 13 can be injected the polymer material prepared for injecting, meaning in liquid or fluid or pasty state.

The injection can be made through a passing opening 15 which is obtained in the convex die 14 and a corresponding hole 16 obtained in the cotyle 1, normally at the polar vertex of this.

To ensure the layer 9 of polymer material adheres completely to the concave surface 7, the latter has roughness in high or bas-relief and the polymer material is allowed to solidify so that the roughness is integrated stably in the layer 9.

The roughness “R” is made in various ways, according to known techniques: for example, it can be made during the forming of the cotyle 1, or else applied afterwards by means of metal powder spraying, or yet again by spreading metal materials or mechanical machining after the cotyle 1 has formed.

According to the invention, different versions of the cotyle 1 can be made that are described in detail below.

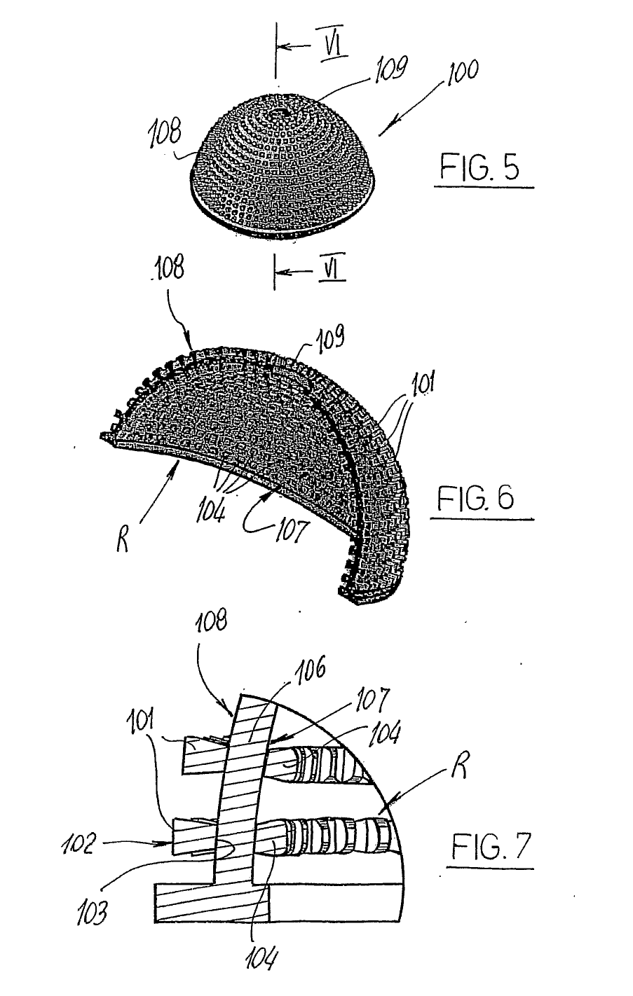

According to a first version shown in the FIGS. 5, 6, 7 it will be noticed that the cotyle, indicated in this case by 100, has a convex surface 108 from which protrude in relief a plurality of teeth 101 that are distributed along meridians and parallels and which have a section shaped substantially like an isosceles trapezium, with the bigger base 102 turned outwards and the smaller base 103 turned towards the convex surface 108 to which it is attached.

The concave counter surface 107 of the cotyle 100 also has teeth 104 in relief which though substantially the same as the previous teeth 101, are turned in the opposite direction to these, meaning with the bigger bases 105 turned towards the inside of the concave surface 107 and the smaller bases 106 turned towards the concave surface 107 to which they are attached.

This way, an outer grid of teeth 101 is formed on the convex surface 108, which favours the grip with the internal bone surface of a cotyloid cavity, and an inner grid of teeth 104 that favours the grip with a layer of coating material, as better indicated below. In the polar area, the cotyle 100 has a through hole 109 to allow the transit of materials ready to create an inner coating of the concave surface 107.

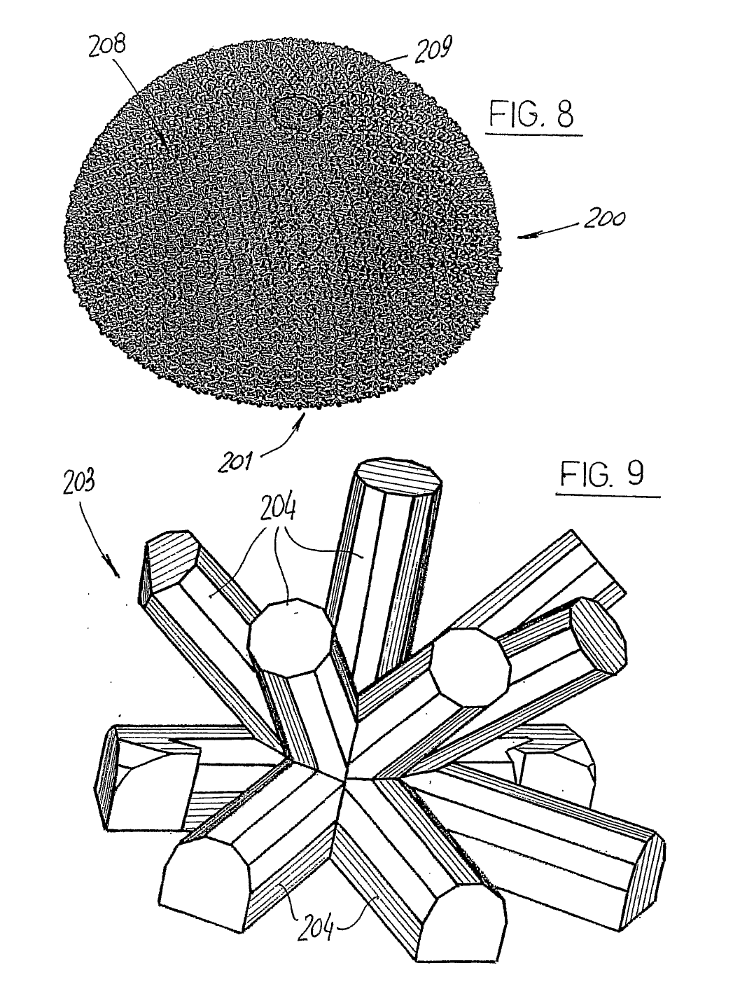

In the second version of the cotyle shown in the FIGS. 8 and 9, it will be seen that the cotyle, indicated in this case by 200, has an external convex surface 208 affected by a relief grid 201.

This relief grid 201 is made up of a plurality of identical elements 202, or substantially such, indicated by 203, one of which is shown in the FIG. 9.

In this Figure, it will be seen that the element 203 comprises a body with several arms 204 which intersect one another reciprocally at 45° angles and ready to engage with the bone portion of a cotyloid cavity of a hip in which the cotyle 200 is designed to be stably fitted without the possibility of moving.

In this second version as well, the cotyle 200 has a substantially cup shape, normally spherical, and has a through hole 209 in the polar area for the same purpose described previously.

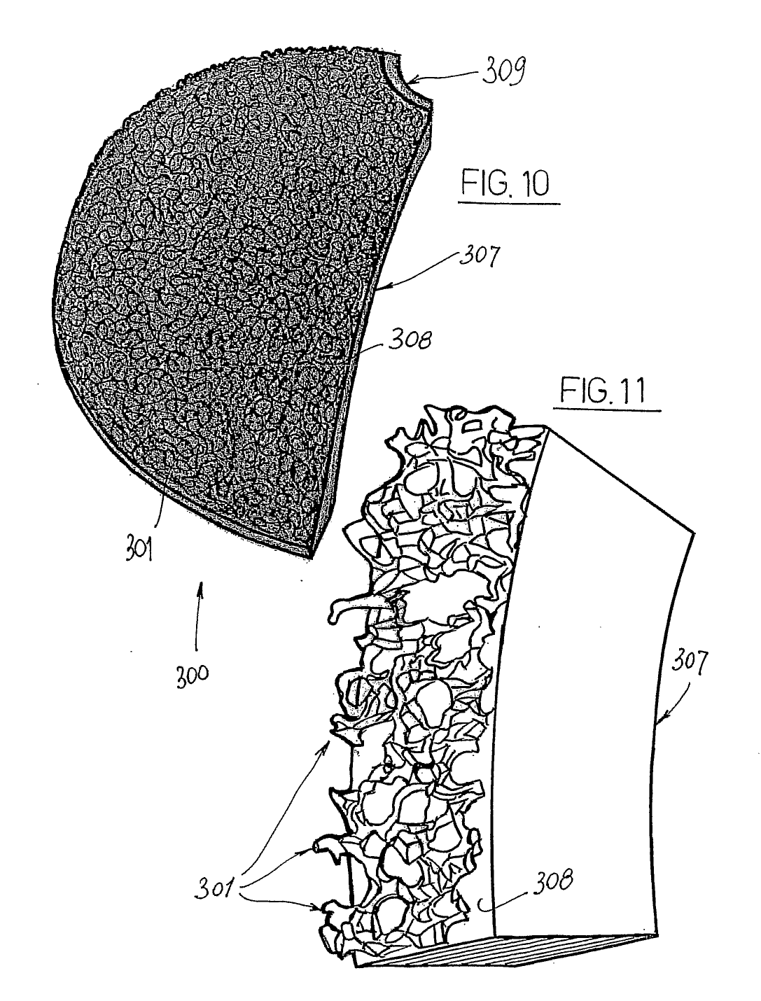

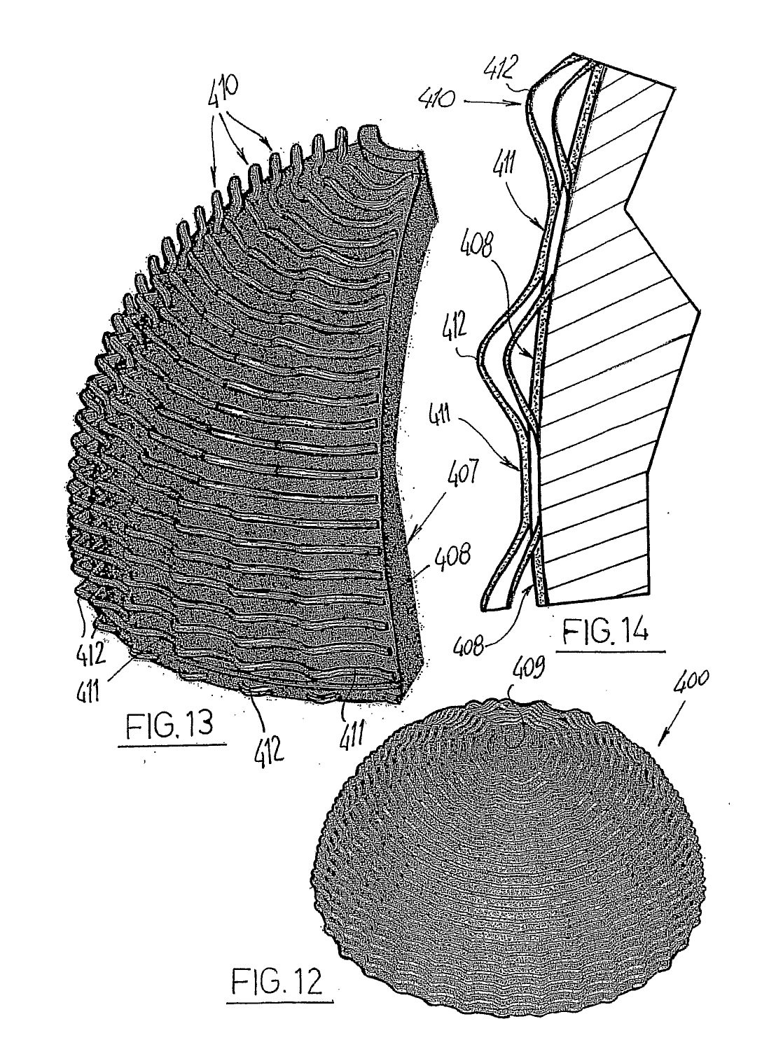

In the third version of the cotyle, indicated in this case by 300, shown in the FIGS. 10 and 11, it will be noticed that it still has a cup shape and has an outer convex surface 308 and an inner concave counter surface 307.

At least the outer surface 308 has a high-relief or bas-relief grid, generally indicated by 301.

The bas-relief, or high-relief 301 substantially reproduces the surface of a bone portion and is suitable, in this case as well, for favouring coupling with the inner walls of a cotyloid cavity without any possibility of reciprocal movements existing.

For this third version of the cotyle 1 as well, a through hole 309 is provided in the polar area for the previously-indicated purposes.

In the fourth version of the cotyle, indicated in this case by 400, shown in the Figures from 12 to 14, it will be noticed that this still has a cup shape, normally spherical, which defines an outer convex surface 408 and a concave counter surface 407.

At least on the convex surface 408 are obtained ribs in relief 410 which have a wavy pattern and which have sections 411 substantially adhering tangentially to the convex surface 408 alternated with sections 412 that are slightly raised from this and protruded outwards.

These raised sections 412 preferably have a substantially convex and pointed shape.

In this fourth version as well, a through hole 409 is provided in the polar area of the cotyle 400.

In the fifth version of the cotyle, indicated in this case by 500, shown in the Figures from 15 to 17, it will be noticed that this still has a cup shape, normally spherical, which defines an outer convex surface 508 and a concave counter surface 507.

At least on the convex surface 508 a grid is obtained made up of cross elements 510 connected the one to the other.

Each cross element 510 has mutilated arms 511 turned outwards and positioned in a substantially radial direction.

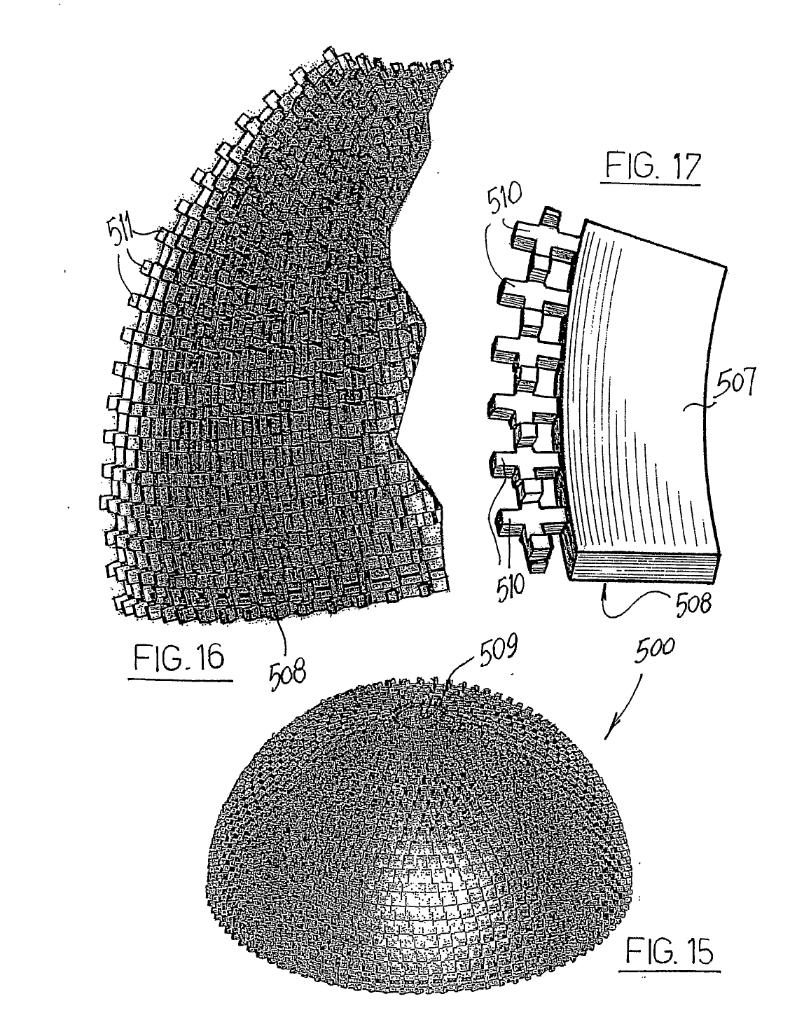

As can be seen in the FIG. 15, in this fifth version as well, a through hole 509 is provided in the polar area of the cotyle 500.

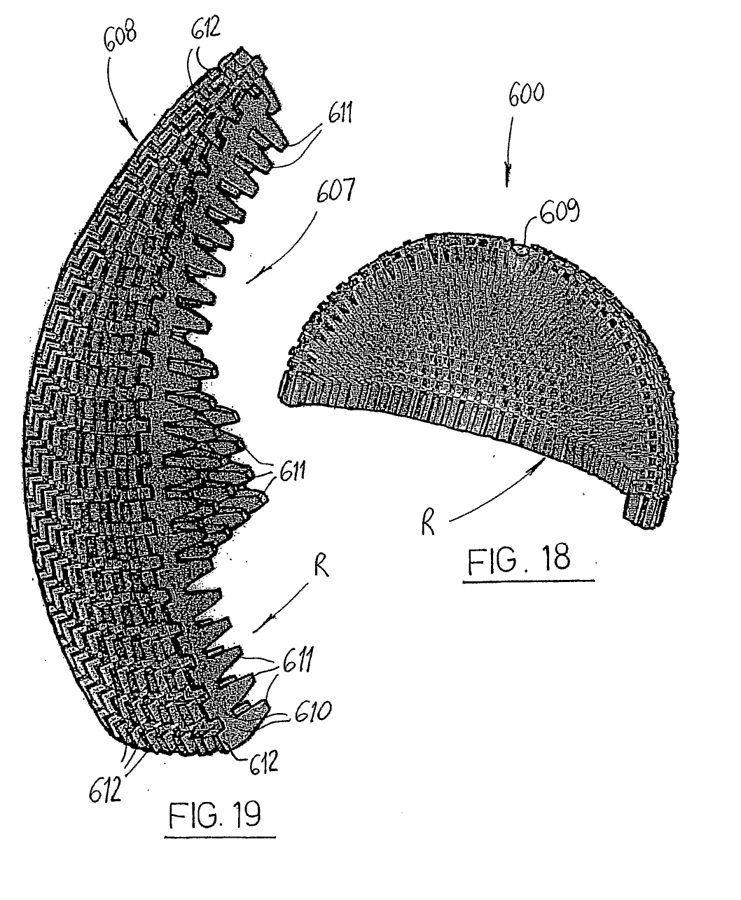

In the sixth version of the cotyle, indicated by 600 and shown in the FIGS. 18 and 19, it will be noticed that this still has a spherical cup shape that defines a concave surface 607 and a convex counter surface 608.

On both these surfaces, relief elements are obtained which are shaped like sorts of teeth 610.

Each tooth 610 comprises a first portion 611 with a substantially truncated pyramid shape which is turned towards the inside of the concave surface 607 and a second counter portion 612 with a substantially parallelepiped shape which extends from the first portion 611 outwards and in relief with respect to the convex surface 608.

Generally speaking, the plurality of teeth 610 creates a multitude of points inside the concave surface 607 turned towards the inside of the cotyle 600 which favour the grip and penetration of the layer of polymer material 9 while the multitude of second portions 612 favours the grip of the bone parts with which the convex surface 608 comes into contact when the cotyle 600 is positioned inside the cotyloid cavity 5.

In this sixth version as well, a through hole 609 is provided in the polar region of the cotyle 600.

In the seventh version of the cotyle 1, indicated in this case by 700 and visible in the Figures from 20 to 22, it will be noticed that this still has a spherical cup shape that defines a concave surface 707 and a convex counter surface 708.

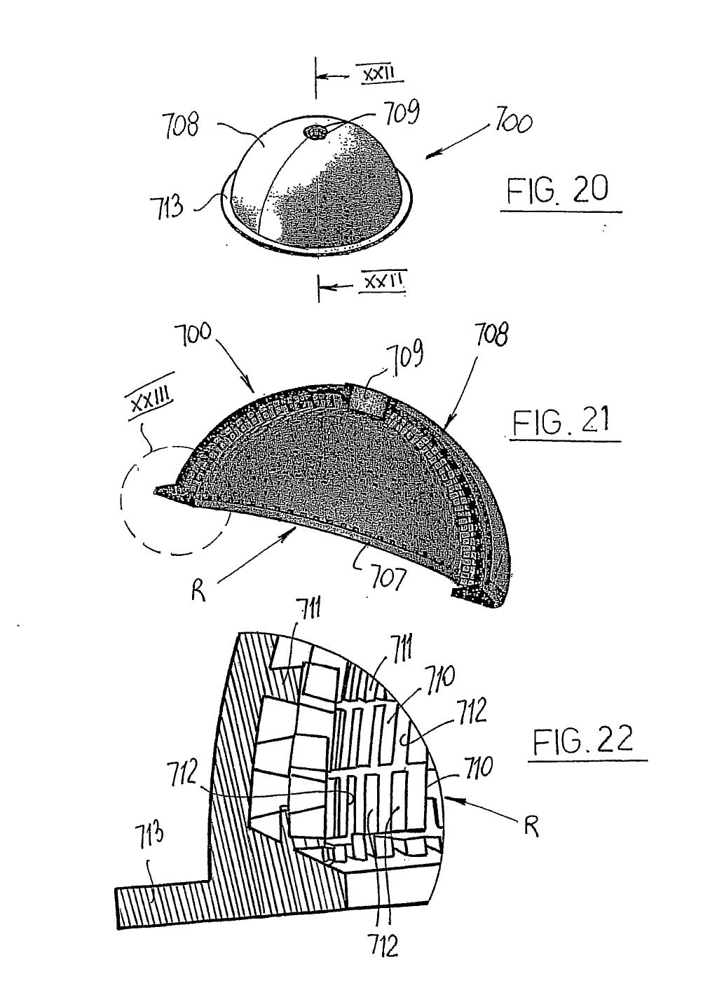

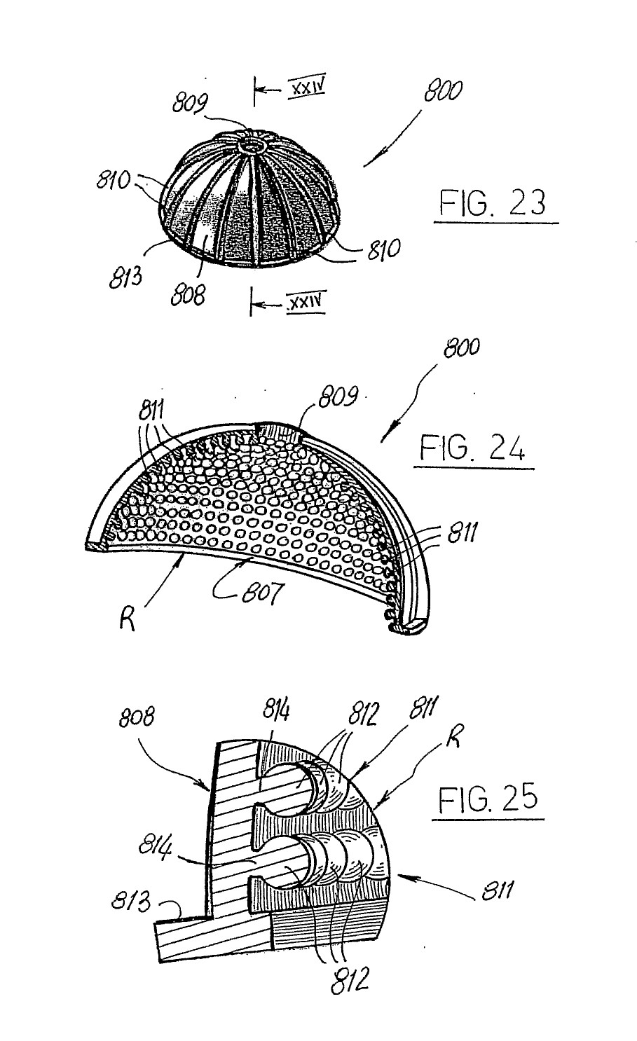

The convex surface 708, as can be seen in the FIG. 20, is substantially smooth, while the concave surface 707 has a grid made ready to favour the grip of the layer of polymer material 9.

This grid, as can be seen in the FIG. 22, is made up of ribs 710 and 711 which are in relief with respect to the concave surface 707 and which intersect, forming slots 712 the inside of which is to be occupied by the layer of polymer material 9, as will be better specified later.

The presence is also noticed of a surrounding edge 713 that surrounds the base of the cotyle 700 in which, as in the other previously-described versions, a through hole 709 is obtained in the polar area.

In the eighth version of the cotyle, indicated in this case by 800, shown in the Figures from 23 to 25, it will be noticed that in this case as well it has the shape of a spherical cup that defines a concave surface 807 and a convex counter surface 808.

The convex surface 808 has a plurality of ribs in relief, indicated by 810 that are arc shaped and are arranged along meridians of the spherical cup shape; these ribs have respective ends that converge on the one side towards the polar area in which is obtained a through hole 809, and counter ends that diverge and which rest with a surrounding edge 813 in relief from the base of the cotyle 800, connecting with this.

The concave surface 807 has a plurality of elements in relief 811 arranged along parallels of the cup shape.

Each element in relief 811 is shaped substantially like a sphere 812 that connects with the concave surface 807 by means of a relative connecting peduncle 814.

The method for coating prosthesis elements that have a concave surface and a corresponding convex counter surface, in the specific case cotyles 1 to make prostheses “P” of the hip, comprises making in a way known to the technician in the field a cotyle which has a spherical cup shape or a substantially spherical shape.

Subsequently, the obtained cotyle 1 is arranged inside a concave die 14 of a mould 11 of a press, in such a way that its concave surface 7 is turned towards the male punch 13.

Both the cotyle 1 and the concave die 14 have a through hole 16 and a passage 15 respectively which, when the cotyle 1 is arranged inside the concave die 14, are aligned with one another, as shown in the FIGS. 2 and 3.

The punch 13 has a coupling surface with the concave surface 7 of the cotyle 1 which is complementary with this and which has a radius of curvature slightly lower than the radius of curvature of the concave surface 7, so that when the punch 13 is moved towards the concave die 14 and couples with this, arranging its complementary profile inside the concave surface 7, an inter-space is defined between these that has a preset thickness: this condition is shown in the FIG. 4.

Through the passage 15 and the through hole 16 a plastic material in mouldable state, normally liquid or very fluid, is then injected under pressure, and this expands in the previously defined inter-space, occupying it completely and coating all the concave surface 7 of the cotyle 1.

Because the roughness “R”, in the various previously described forms, has previously been obtained in this concave surface 7, these are automatically integrated in the coating layer 9 which forms when the material that has been injected into the inter-space is allowed to solidify.

This way, the layer 9 becomes a single body with the cotyle 1 and it is no longer possible to separate it from this.

The thickness of the inter-space can be changed according to need, in such a way as to obtain coatings 9 of required thicknesses.

It should be noticed that roughness “R” can also be obtained on the outer surface of the cotyle 1, during the forming of this, which can favour the fixed fitting of the cotyle 1 inside a cotyloid cavity 5 without any reciprocal movements occurring.

1. A method for coating prosthesis elements that have a concave surface and a corresponding convex counter surface, comprising:
arranging in at least said concave surface shaped retentive means for gripping coating means;
coupling coating means with said shaped retentive means, wherein said coupling comprises coating said concave surface with said coating means temporarily in a mouldable state to obtain a moulded coating means;
making said moulded coating means solidify so that it adheres firmly to said shaped retentive means.

2. The method according to claim 1, wherein said coating comprises making said retentive means and said coating means reciprocally penetrate the other.

3. The method according to claim 1, wherein said moulded coating means comprises a layer polymer material which defines a low friction concave face.

4. The method according to claim 1, wherein said retentive means comprises high and/or bas-relief elements applied at least on said concave surface.

5. The method according to claim 4, wherein said high or bas-relief elements comprise grids formed with elements chosen from at least one of teeth in relief, bodies with several arms intersecting one another reciprocally, roughness that reproduces bone structures, wavy ribs, cross elements, sorts of teeth having first portions and second portions connected with each other, ribs substantially straight intersecting one another, arc-shaped ribs, and spheres in relief.

6. The method according to claim 1, wherein said coating comprises placing a grid means between said concave surface and said coating means such a way that said grid means are integrated in said coating means.

7. The method according to claim 1, wherein said coating comprises coating inside a mould.

8. The method according to claim 7, wherein said coating inside a mould comprises: positioning said prosthesis elements inside respective dies; introducing punches having convex surfaces that shape complementary profiles of said concave surfaces inside of these; after said introducing, keeping an inter-space between said complementary profiles and said concave surfaces; filling said inter-space with said coating means prepared in said mouldable state.

9. The method according to claim 8, wherein said filling comprises injecting under pressure.

10. The method according to claim 8, wherein said inter-space has a substantially constant and predeterminable thickness.

11. The method according to claim 3, wherein said polymer material is a biologically compatible polymer chosen from UHMWPE (ultra-high molecular weight polyethylene), cross-linked UHMWPE, Polyurethane, PEEK (polyetheretherketone), carbon fibre reinforced PEEK (polyetheretherkone).

12. The method according to claim 1 wherein said prosthesis elements comprise cotyles to make a prosthesis of the hip.

13. A prosthesis element, comprising:
a concave surface;
a layer of low friction coating material arranged on said concave surface and coupled with a natural or artificial joined convex surface of a bone body, said coating layer being penetrated by retentive means obtained on said concave surface.

14. The element according to claim 13 wherein said prosthesis elements comprises cotyles and wherein said convex surface comprises a head of a thigh bone or a shape thereof.

15. The element according to claim 14, wherein said cotyles have a concave surface and a convex counter surface, at least said concave surface being provided with said retentive means.

16. The element according to claim 13, wherein said retentive means includes high or bas-relief elements applied at least on said concave surface.

17. The element according to claim 16, wherein said retentive means is chosen from at least one of: teeth in relief, bodies with several arms reciprocally perpendicular, roughnesses that reproduces bone structures, wavy ribs, cross elements, sorts of teeth having first portions and second portions connected with each other, ribs substantially straight intersecting one another, arc-shaped ribs, and spheres in relief.
